(19) United States
(12) Patent Application Publication
(10) Pub. No.: US 2026/0198964 A1
(43) Pub. Date: Jul. 16, 2026

(54) SELF-HEALING SEAL AND CONNECTOR PORT

(71) Applicant: Terumo Corporation, Tokyo (JP)

(72) Inventors: Melissa Buzby, Long Beach, CA (US); Bao Khuu, Irvine, CA (US); Gilberto Caudillo Barba, Rancho Santa Margarita, CA (US); Frank Bernard, Dana Point, CA (US); Brian Gray, Trabuco Canyon, CA (US)

(21) Appl. No.: 19/130,737

(22) PCT Filed: Nov. 17, 2023
(86) PCT No.: PCT/US2023/080289

§ 371 Date: May 16, 2025

Related U.S. Application Data
(60) Provisional application No. 63/426,236, filed on Nov. 17, 2022.

(51) Int. Cl. A61B 17/34; A61B 17/22

(52) U.S. Cl. CPC A61B 17/3462; A61B 17/3498; A61B 2017/22079

(57) ABSTRACT

A self-healing seal and connector port are provided with a body including a first port, a second port, and a third port; a cap, secured to the third port; and a self-healing seal secured to the third port by the cap, wherein the self-healing seal has a circular face and includes: a first ring located on an edge of the circular face, captured between the body and the cap; and a second ring, located centrally on the circular face, including a raised ridge on a first side of the circular face defining an inverted conic bevel, and including a tearing guide on a second side of the circular face centered on the inverted conic bevel.

### CROSS-REFERENCES TO RELATED APPLICATIONS

The present disclosure claims the benefit of U.S. Provisional Patent Application No. 63/426,236 entitled “SELF-HEALING SEAL AND CONNECTOR PORT” and filed on 2022 Nov. 17, which is incorporated herein by reference in its entirety.

### BACKGROUND

When aspirating body fluids and any solids carried therewith (e.g. embolisms in blood), a negative pressure is held on a fluid target (e.g., a vein) to draw the fluids and solids out. A catheter carries these captured fluids and solids to a port, but for continued negative pressure to be applied, the solids and liquids may need to be periodically removed to avoid clogging the negative pressure source or identifying when a solid has successfully been extracted from the fluid target.

### SUMMARY

The present disclosure is generally related to a self-healing connector seal and port(s) that allow a user to insert a needle or other instrument through the connector (e.g., to unclog or extract solids from a port used with a catheter for aspiration) and then remove the needle or other instrument while maintaining negative pressure on the fluid target. The connector port and the seal include several features to ensure that insertion of the needle or instrument leaves a clean hole in the seal so that a negative pressure may be continuously applied as the needle or instrument are inserted and retracted.

One embodiment of the present disclosure is a device, comprising: a body including a first port, a second port, and a third port; a cap, secured to the third port; and a self-healing seal secured to the third port by the cap, wherein the self-healing seal has a circular face and includes: a first ring located on an edge of the circular face, captured between the body and the cap; and a second ring, located centrally on the circular face, including a raised ridge on a first side of the circular face defining an inverted conic bevel, and including a tearing guide on a second side of the circular face centered on the inverted conic bevel.

One embodiment of the present disclosure is a self-healing seal, comprising: a first ring located on an edge of a circular face; and a second ring, located centrally on the circular face, including a raised ridge on a first side of the circular face and defining an inverted conic bevel, and including a tearing guide on a second side of the circular face centered on the inverted conic bevel.

One embodiment of the present disclosure is a device, comprising: a body including a first port, a second port, and a third port; a sealing means connected to the third port that is configured to maintain a seal when a pressure is applied to the first port through the second port, the sealing means including: a first ring located on an edge of a circular face; and a second ring, located centrally on the circular face, including a raised ridge on a first side of the circular face that defines an inverted conic bevel, and includes a tearing guide on a second side of the circular face centered on the inverted conic bevel.

### BRIEF DESCRIPTION OF THE DRAWINGS

The accompanying figures depict various elements of the one or more embodiments of the present disclosure, and are not considered limiting of the scope of the present disclosure.

In the Figures, some elements may be shown not to scale with other elements so as to more clearly show the details. Additionally, like reference numbers are used, where possible, to indicate like elements throughout the several Figures.

It is contemplated that elements and features of one embodiment may be beneficially incorporated in the other embodiments without further recitation or illustration. For example, as the Figures may show alternative views and time periods, various elements shown in a first Figure may be omitted from the illustration shown in a second Figure without disclaiming the inclusion of those elements in the embodiments illustrated or discussed in relation to the second Figure.

FIGS. 1A-1C provide views of an assembled device, according to embodiments of the present disclosure. FIGS. 1A and 1C are cross-sectional views of single-valve and dual-valve embodiments, respectively. FIG. 1B is a top view of an assembled device.

FIG. 2 provides a cross-sectional view of a body for use in the device, according to embodiments of the present disclosure.

FIG. 3 provides a cross-sectional view of a cap for use in the device, according to embodiments of the present disclosure.

FIGS. 4A-4G provide views of a self-healing seal, according to embodiments of the present disclosure.

FIGS. 5A-5C provide cross-sectional views a self-healing seal in various states of operation, according to embodiments of the present disclosure.

### DETAILED DESCRIPTION

The present disclosure is generally related to an improved syringe plunger lock for use for use with a syringe and plunger system as part of a suction or aspiration device. The improvements described herein provide various benefits, including, but not limited to: easier use of the associated devices, more precise control of suction generated by manual aspiration system, and improved or simplified manufacturing techniques.

The described system includes a rotational lock to secure the plunger at a known position in the syringe to maintain vacuum more easily and precisely. Ribs on the plunger can pass around a locking tooth on the rotational lock when drawn outward, and prevent the plunger from traveling inward to the syringe by the resulting negative pressure until a user manually releases the lock.

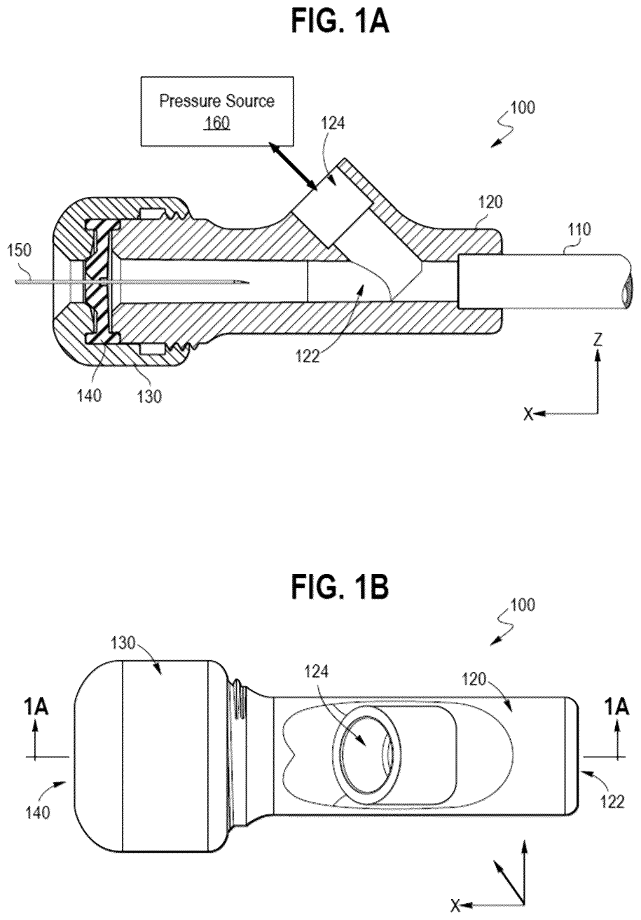

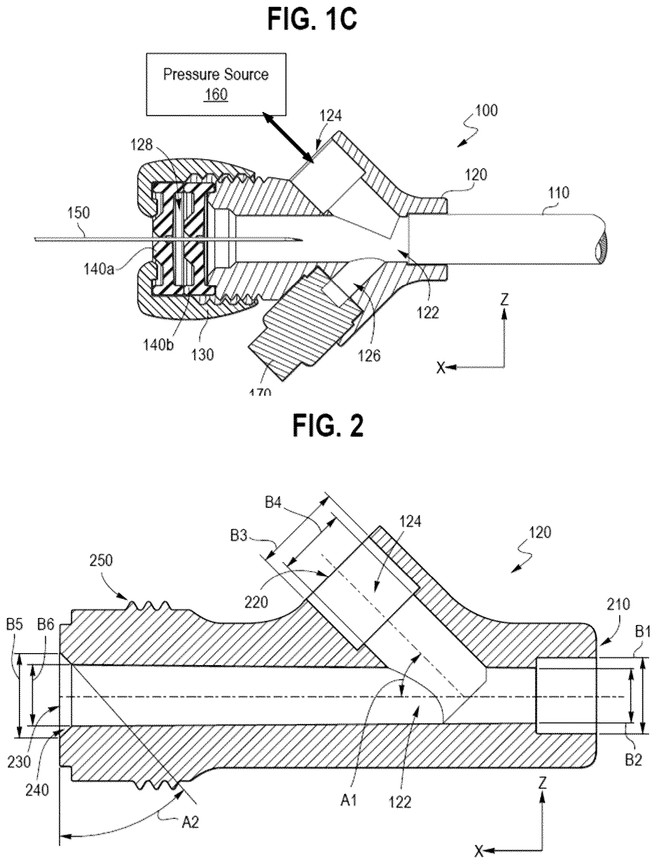

FIGS. 1A-1B provide views of an assembled port adapter 100, according to embodiments of the present disclosure. FIG. 1A provides a cross-sectional view of the port adapter 100, and FIG. 1B provides a planar view of the port adapter 100.

In FIG. 1A, a catheter 110 is secured in the body 120, and an instrument 150 (e.g., a dilator or guide wire) is inserted through a self-healing seal 140 held in place to the body 120 via a cap 130. The body 120 defines a first passage 122 through which the instrument 150 is guided into (and out of) the catheter 110 and a second passage 124 that connects the first passage 122 (and a fluid target in fluid communication with the catheter 110) with a pressure source 160, such as, for example, a vacuum pump or syringe. The self-healing seal 140 acts as a sealing means to the port adapter 100 that allows for the insertion and removal of the instrument 150 without loss of effective pressure application on the fluid target. Stated differently, the self-healing seal 140 operates in both an intact and punctured state to isolate the first passage 122 from ambient air so that the pressure source 160 exerts pressure on the fluid target and does not draw in excess air through the first passage 122.

In FIG. 1B, the port adapter 100 is shown in a planar view perpendicular to the cross-sectional view in FIG. 1A. The cap 130 is secured on one end of the body 120, opposite to the end in which the catheter 110 is secured, and obscures the self-healing seal 140 in the view shown in FIG. 1A. The first passage 122 runs the longitudinal length of the port adapter 100, while the second passage 124 is oriented in a non-parallel plane to intersect with the first passage 122, thereby placing the two passages in fluid communication with one another.

In FIG. 1C, a catheter 110 is secured in the body 120, and an instrument 150 (e.g., a dilator or guide wire) is inserted through a first self-healing seal 140a and a second self-healing seal 140b that are held in place to the body 120 via a cap 130. The body 120 defines a first passage 122 through which the instrument 150 is guided into (and out of) the catheter 110 and a second passage 124 that connects the first passage 122 (and a fluid target in fluid communication with the catheter 110) with a pressure source 160, such as, for example, a vacuum pump or syringe.

An additional third passage 126 (with an associated fourth port) is also illustrated, with is secured with a Luer activated valve 170, which selectively seals off the passages from the external environment, but allows an operator to inject fluids (e.g., dyes, saline, contrasts) into the system. The first and second self-healing seals 140a-b act as a sealing means to the port adapter 100 that allows for the insertion and removal of the instrument 150 without loss of effective pressure application on the fluid target. Stated differently, the self-healing seal 140 operates in both an intact and punctured state to isolate the first passage 122 from ambient air so that the pressure source 160 exerts pressure on the fluid target and does not draw in excess air through the first passage 122. The use of two (or more) self-healing seals 140 provides for an airlock 128 between a pair of self-healing seals 140. This airlock 128 provides for a medial pressure differential (e.g., between external air pressure and airlock pressure, and between airlock pressure internal passage pressure), which allows a pair of self-healing seals 140 to maintain a seal when a greater pressure differential is applied by the pressure source 160 that a single self-healing seal 140 may be able to provide.

The volume of the airlock 128 is determined by the geometry of the two self-healing seals 140. For example, when using two self-healing seals 140 with a height (shown in the X direction in FIG. 1C) of 0.20 inches, distances from the top and bottom of the outer ring to the circular face each of 0.065 inches, an outer diameter (shown in the Y direction in FIG. 1C) of 0.715 inches, and a thickness of the outer ring (shown in the Y direction in FIG. 1C) of 0.06 inches, the volume would be approximately 0.07 cubic inches. As will be appreciated, this approximate volume does not account for any volume occupied by an inserted instrument 150, or the relative volumes occupied or vacated by the raised ridges and tear-guiding features of the self-healing seals 140.

FIG. 2 provides a cross-sectional view of a body 120 for use in the device, according to embodiments of the present disclosure. The body 120 includes a first port 210, a second port 220, and a third port 230 that are in fluid communication with one another via the first passage 122 and the second passage 124. The first port 210 is aligned on a shared axis with the third port 230 along the longitudinal length of the body 120 and between which the first passage 122 runs, while the second port 220 is disposed between the first port 210 and the third port 230 at an intersecting angle A1 with the shared longitudinal axis to connect the second passage 124 with the first passage 122. As illustrated, the intersecting angle A1 is 45 degrees, but in various embodiments may include other angles, including 90 degrees, 60 degrees, 30 degrees, etc.

The first port 210 includes a first bore B1 and a second bore B2 of different sizes from one another. In various embodiments, the first bore B1 is sized to allow insertion of a catheter 110, while the second bore B2 is smaller than the diameter of the catheter 110 to prevent or restrict over-insertion of the catheter 110 into the first passage 122. In various embodiments, the size of the first bore B1 is configured to engage an inserted catheter 110 via friction in a cavity having a substantially cylindrical shape with a diameter of the first bore B1, although various adhesives or weld joints or other connectors may be used in addition or alternatively to friction fitting. The first bore B1 matches the inner diameter of the shaft, which reduces the possibility of restrictions and Venturi effects of the overall through-diameter, which thereby reduces the risk of clogs during aspiration due to turbulent flows at the restricted portions.

The second port 220 includes a third bore B3 and a fourth bore B4 of different sizes from one another. In various embodiments, the third bore B3 is sized to allow insertion of a pneumatic tube connected to a pressure source 160, while the fourth bore B2 is smaller than the diameter of the tube to prevent or restrict over-insertion of the tube into the second passage 124. In various embodiments, the size of the third bore B3 is configured to engage an inserted tube via friction (or adhesives, weld joints, or other connectors) in a cavity having a substantially cylindrical shape with a diameter of the third bore B3.

The third port 230 includes a fifth bore B5 and a sixth bore B6 of different sizes from one another. The third port 230 is where the self-healing seal 140 mates to the body 120, and the fifth bore B5 is larger than the sixth bore B6, and reduces to the sixth bore B6 via an inward chamfer 240. As illustrated, the reducing angle A2 of the inward chamfer 240 is approximately 45 degrees, but in various embodiments may include other angles between 0 and 90 degrees, including 45 degrees, 60 degrees, 30 degrees, 15 degrees, etc.

The chamfers 240 contribute to forming a seal when pressure is applied after a through-hole is formed through the self-healing seal 140. For example, while the system is under vacuum, the self-healing seal 140 may be pulled against the chamfer 240 and is thereby compressed or “squeezed” to fill the reduced volume. This compression of the self-healing seal 140 against the chamfer 240 causes any tears or gaps (e.g., those caused by one or more inserted instruments) to be forced closed by the body of the self-healing seal 140 so as to close those tears and gaps and thereby maintain the seal under vacuum pressure. Additionally, the inward chamfer 240 may also guide deflection of the self-healing seal 140 to reduce undesired tearing when an instrument 150 punctures the self-healing seal 140 and to ensure proper closure of any puncture when the instrument 150 is removed. Although described in the present disclosure as “chamfers” illustrated as having substantially flat surfaces having a given angle relative to a longitudinal axis of the port adapter 100 (when assembled), the described chamfers may also refer to curved bevels or a series of straight chamfers having multiple different angles defined over the series of straight chamfers.

The body 120 additionally includes threads 250 disposed on the side of the body 120 that includes the third port 230, which are provided to interface and secure a cap 130 to the body 120, thereby holding the self-healing seal 140 in place.

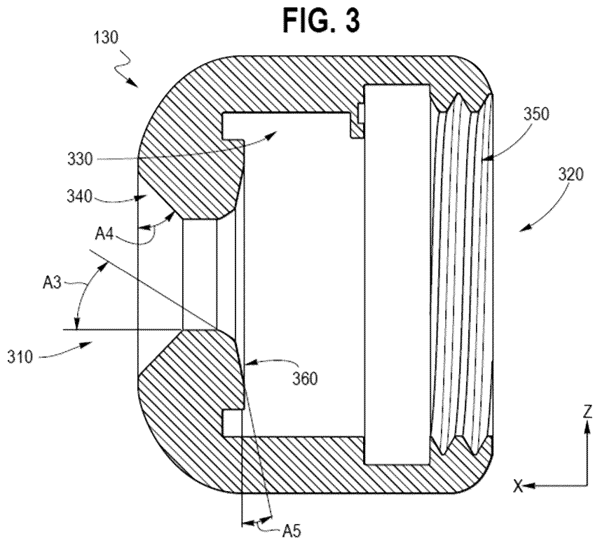

FIG. 3 provides a cross-sectional view of a cap 130 for use in the port adapter 100, according to embodiments of the present disclosure. The cap 130 is provided to secure the self-healing seal 140 to the body and guide inserting of an instrument 150 through the self-healing seal 140. The cap 130 includes a first opening 310 on a first side, and a second opening 320 on an opposite side. A holding cavity 330 is defined between the first opening 310 and the second opening 320 to hold the self-healing seal 140 between a cavity 330 in the cap 130 and the third port 230.

The first opening 310 includes an inward chamfer 340 having a fourth angle A4 to help guide the instrument 150 during insertion. As illustrated, the fourth angle A4 is 45 degrees, but in various embodiments may include other angles, including 60 degrees, 30 degrees, 15 degrees, etc. The first opening also includes an outward chamfer 360 having a fifth angle A5 to affect the amount of outward flexion (e.g., towards the first opening 310 versus towards the second opening 320) that the self-healing seal 140 is allowed to allowed to bend, thereby affecting where the self-healing seal 140 bends to affect a closure of a puncture through the self-healing seal 140. As illustrated, the fifth angle A5 is 10 degrees, but in various embodiments may include other angles, including 5 degrees, 15 degrees, 30 degrees, etc.

The second opening 320 includes threads 350 to interface the threads 250 defined on the body 120 to hold the cap 130 and the self-healing seal 140 in place against the body 120. The second opening 320 is sized to interface with the third port 230 of the body 120 and allow insertion of the self-healing seal 140 into the cavity 330. Accordingly, a user may initially insert the self-healing seal 140 into the cavity 330 via the second opening 320 and screw the cap 130 onto the body 120 via the associated threads 250/350. Similarly, a user may remove the cap 130 from the body 120 by unscrewing the associated threads 250/350 from one another, and may remove the self-healing seal 140 from the cavity 330 via the second opening 320 (e.g., to replace the self-healing seal 140).

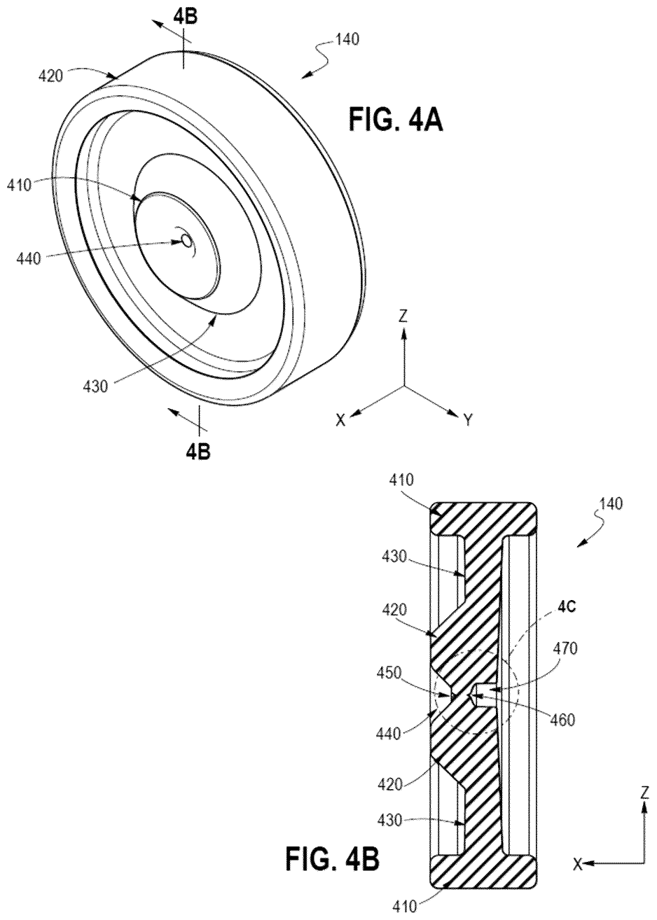

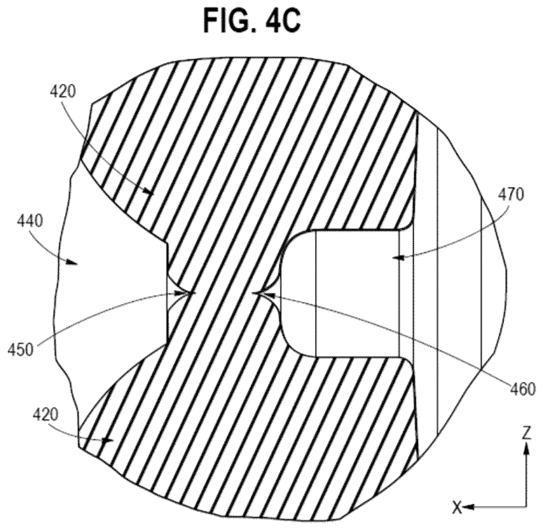

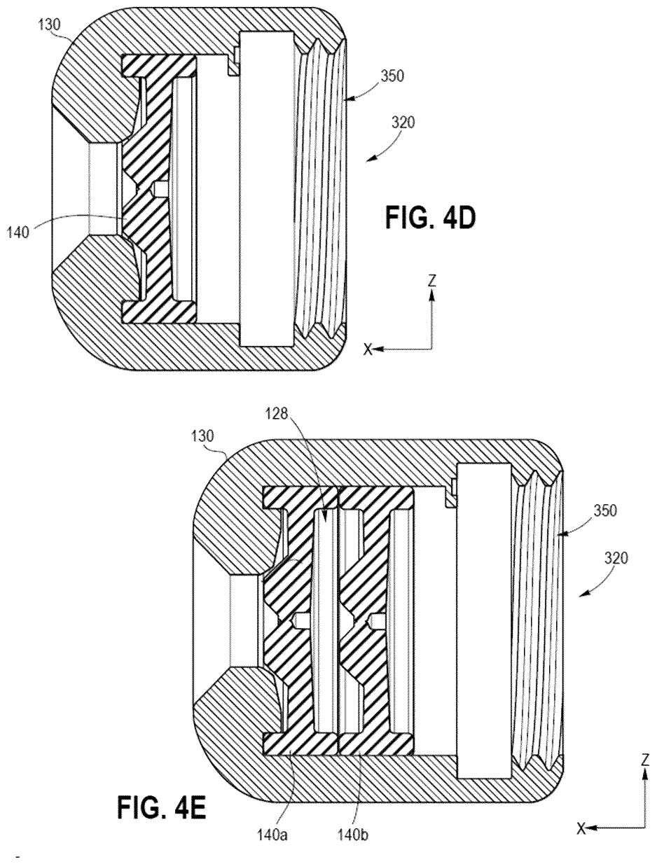

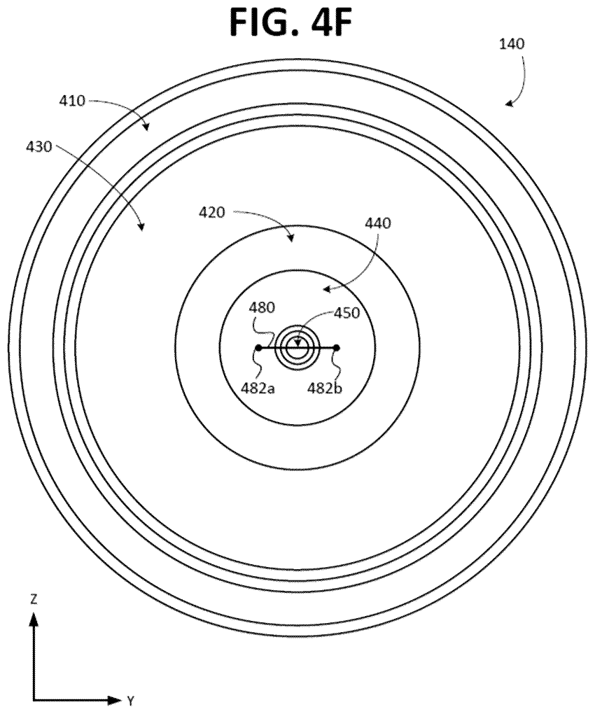

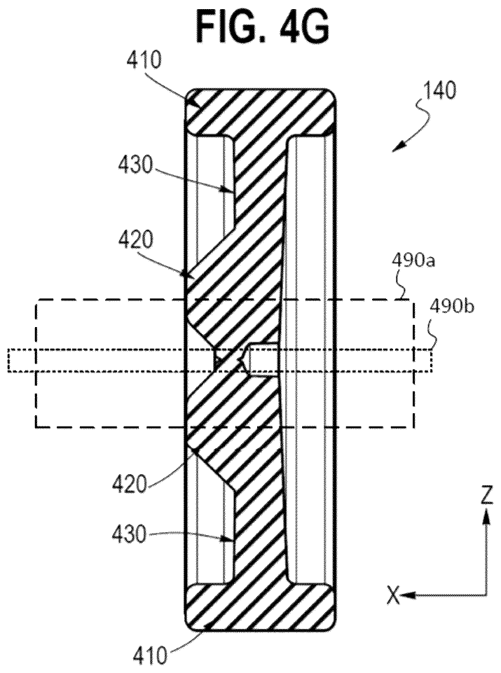

FIGS. 4A-4F provide views of a self-healing seal 140, according to embodiments of the present disclosure. FIG. 4A provides an isometric view of a self-healing seal 140, FIG. 4B provides a cross-sectional view of the self-healing seal 140, FIG. 4C provides a detailed view of the tearing guides, FIG. 4D provides a cross-sectional view of the self-healing seal 140 inserted into the cavity 330, according to embodiments of the present disclosure, FIG. 4E provides a cross-section view of a pair of self-healing seals 140 inserted into the cavity 330, FIG. 4F provides a planar view of a self-healing seal 140, and FIG. 4G illustrates puncturing pathways through a self-healing seal 140.

In various embodiments, the self-healing seal 140 is made of various flexible materials that allow for cap 130 and the body 120 to compress the self-healing seal 140 (thereby forming a seal around the outer circumference of the self-healing seal 140) and for an applied pressure bend or collapse the self-healing seal towards a lower pressure side. For example, the self-healing seal 140 may be made of various rubbers, silicones, nylons, and other materials, which are selected to have a high elasticity and a low durometer. In various embodiments, the material being selected to have a “high elasticity” refers the material having an elasticity ratio of at least 400%, preferably of at least 500%, more preferably of at least 600%, and even more preferably of at least 700%. In various embodiments, the material being selected to have a “low durometer” refers the material having a Shore hardness of 55 A or below, preferably of 50A or below, more preferably of 40A or below, and even more preferably of 30A or below.

Various features of the self-healing seal 140 control the size and location of any induced through-holes and provide for the controlled collapse of the self-healing seal 140 around that through-hole when an instrument is removed-thereby sealing the through-hole.

As shown in FIGS. 4A-4F, the self-healing seal 140 has a substantially circular face and includes a first ring 410 that located on an outer edge of the circular face extending from a first side and a second side of the self-healing seal 140. In various embodiments, the first ring 410 extends to an equal prominence relative to the first and second sides of the self-healing seal 140, but may extend to unequal prominences in other embodiments.

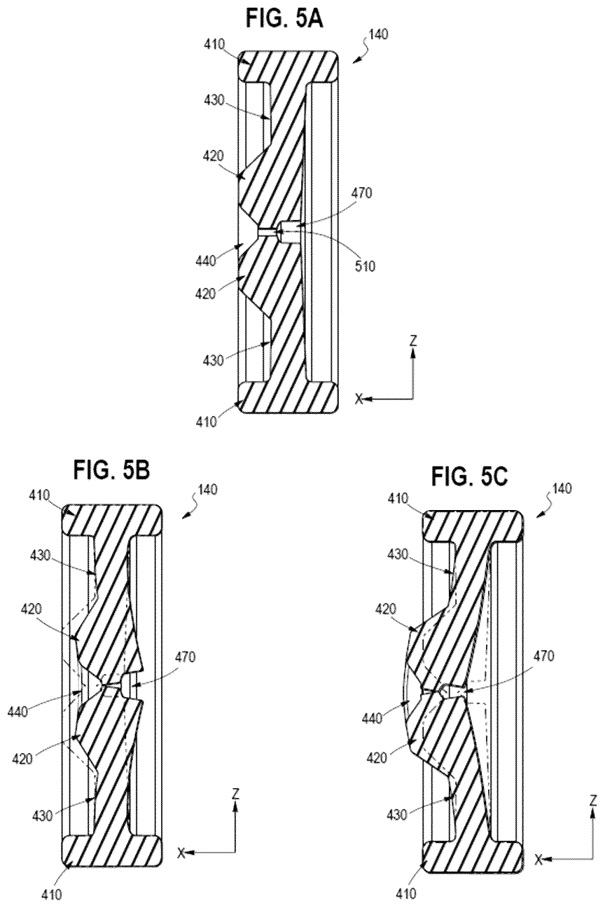

The self-healing seal 140 also includes a second ring 420 that is located centrally on the first side, and a tearing guide 470 that is located centrally on the second side. The second ring 420 includes a raised ridge that defines an inverted conic bevel 440. The conic bevel 440 is aligned with (e.g., centered on) the tearing guide 470 on the second side, which provide sealing surfaces after a through-hole is made through the self-healing seal 140 (see e.g., FIGS. 5A-5C). Additionally, the conic bevel 440 helps direct the instrument 150 to the center of the self-healing seal 140 during insertion, which in some embodiments includes a first dimple 450 on the first side and a second dimple 460 on the second side, although in some embodiments one or both of the first dimple 450 and the second dimple 460 may be omitted. The first dimple 450 and/or the second dimple 460 define a region of reduced thickness in the self-healing seal 140 that may be more readily pierced by the instrument 150 during insertion, and which allow for a through-hole to be formed with less tearing in other directions, thereby reducing the size of the through-hole to be sealed.

A third ring 430 of a reduced thickness (relative to the thicknesses of the first ring 410 and the second ring 420 in the X direction) is located between the first ring 410 and the second ring 420. The reduced thickness of the third ring 430 encourages flexion of the self-healing seal 140 to occur more readily in the third ring 430 than the other rings 410/420.

The second ring 420 has a foot based on the third ring 430, so that the second ring 420 rises above the third ring 430 and extends to a peak (in the X direction) that is equal to the peak of first ring 410 (from the first side). The nadir of the inverted conic bevel 440 is positioned above or the peak of the foot of the second ring 420 (e.g., the inverted conic bevel 440 does not extend past a plane defined by the third ring 430 on the first side of the self-healing seal 140 when in a neutral position). In contrast, the tearing gu4Dide 470 extends inward from the face of the third ring 430 on the second side of the self-healing seal 140 and extends the second dimple 460 to be even with the plan defined by the third ring 430 on the first side of the self-healing seal 140 when in a neutral position.

In various embodiments, the self-healing seal 140 is made of various rubbers or plastics to allow the self-healing seal 140 to bend, flex, or otherwise deform when pushed or pulled by a pressure exerted by the pressure source 160 in the first passage 122. FIG. 4D illustrates a neutral position for the self-healing seal 140 when the pressure exerted on the first side and on the second side are substantially equal. In various embodiments, the self-healing seal 140 is designed to operate with various pressures to reestablish a seal after a through-hole is introduced through the self-healing seal 140 by partially collapsing over the through-hole to prevent or reduce the amount of outside air entering the first passage 122 through the self-healing seal 140 or the amount of internal air (or other fluid) exiting the first passage 122 through the self-healing seal 140. In various embodiments, the self-healing seal 140 is configured to operate at imparted pressures of ±20 pounds per square inch (psi), ±40 psi, or the like.

In various embodiments, the cap 130 is sized to accommodate two or more self-healing seals 140, such as a first self-healing seal 140a and a second self-healing seal 140b illustrated in FIG. 4E. FIG. 4E illustrates a neutral position for two self-healing seals 140a-b. Each of the self-healing seals 140a-b are positioned to be concentrically aligned on a shared axis (e.g., coaxially aligned) to allow insertion of an instrument 150 through the respective inverted conic bevels 440 and tearing guides 470 to create a centrally aligned through-hole. The self-healing seals 140a-b are configured to interface with each other via the respective outer first rings 410 to define an airlock 128 between the inner surfaces (e.g., the second side of the first self-healing seal 140a and the first side of the second self-healing seal 140b) to provides for a medial pressure differential (e.g., between external air pressure and airlock pressure, and between airlock pressure internal passage pressure), which allows a pair of self-healing seals 140a-b to maintain a seal when a greater pressure differentials are applied than a single self-healing seal 140 may be able to provide.

The pair of self-healing seals are designed to operate with various pressures to reestablish a seal after a through-hole is introduced through the pair of self-healing seals 140a-b by partially collapsing over the through-holes to prevent or reduce the amount of outside air entering the airlock 128 through the first self-healing seal 140a and first passage 122 through the second self-healing seal 140b or the amount of internal air (or other fluid) exiting the first passage 122 through the self-healing seals 140a-b. In various embodiments, the pair of self-healing seal 140 is configured to operate at imparted pressures of +40 psi, +80 psi, or the like.

In various embodiments, the paired self-healing seals 140a-b are identical in design, although in some embodiments, the first self-healing seal 140a may be made of a different material, with different dimensions, or combinations thereof, than the second self-healing seal 140b.

Although generally discussed for use with an instrument 150 with a substantially circular cross-section that self-pierces the self-healing seal 140 (e.g., a needle with a sharpened tip), in various embodiments, and as shown in FIG. 4F, the self-healing seal 140 may include a puncturing slit 480 to aid in allowing other devices to be inserted through the self-healing seal 140. The puncturing slit 480 is a non-circular feature that is centered in the self-healing seal 140, which may define a region of reduced thickness in the self-healing seal 140 adapted for a non-circular instrument 150, a circular instrument 150 of a greater diameter than the first dimple 450, a circular instrument 150 without a cutting tip, or an auxiliary tool (e.g., a razor), to pierce the self-healing seal 140 in a regular and controllable manner (e.g., to reduce tearing of the circular face outside of the region when forming a through-hole).

As illustrated in FIG. 4F, the puncturing slit 480 extends for a length (shown in the Y direction) that includes portions within the conic bevel 440, but may extend for different lengths (greater or lesser) in various embodiments than that shown. When extending into portions of the self-healing seal 140 with different prominences (e.g., heights in a Z direction), the puncturing slit 480 may extend for a uniform depth from the surface or to a uniform position defined on the Z axis. In various embodiments, the puncturing slit 480 may be a score line that does not fully penetrate the self-healing seal 140 (until at instrument is passed through) or may be manufactured to penetrate the self-healing seal 140 without the instrument 150 (e.g., providing a pre-formed through-hole).

Although illustrated in FIG. 4F on the first side of the self-healing seal 140, in some embodiments, the puncturing slit 480 may instead be defined on the second side of the self-healing seal 140, or a pair of puncturing slits 480 may be included on opposing sides of the self-healing seal 140 (aligned with one another). In various embodiments, one or more puncturing slits 480 may be included in addition to or instead of one or both of the first dimple 450 and the second dimple 460.

In various embodiments, the puncturing slit 480 terminates with tear arrestors 482a-b on either end, which reduce the likelihood of a through-hole opened in the self-healing seal 140 extending past the edges of the puncturing slit 480. The tear arrestors 482a-b define a region of reduced thickness in the self-healing seal 140 of a greater width than the main length of the puncturing slit 480 (e.g., circular holes or voids in the material of the self-healing seal 140 of greater diameter than the height (shown in FIG. 4F in the Z direction) than the rest of the puncturing slit 480).

FIG. 4G illustrates puncturing pathways 490a-b (generally or collectively, puncturing pathways 490) through a self-healing seal 140. Depending on the size and cross-sectional shape of the instrument 150 to be inserted through the self-healing seal 140, the puncturing pathway 490 may be of different sizes and shapes to accommodate the instrument 150. For example, a razor may be inserted through the first puncturing pathway 490a guided by the puncturing slit 480 to create a through-hole through which various instruments 150 may be inserted. In another example, a needle with a sharpened end may be inserted through the second puncturing pathway 490b guided by the first dimple 450 to create a through-hole through which various instruments 150 may be inserted.

FIGS. 5A-5C provide cross-sectional views a self-healing seal 140 in various states of operation, according to embodiments of the present disclosure. In FIGS. 5A-5C, the first ring 410 remains stationary, held in place by the cap 130 and the body 120 (not illustrated in FIGS. 5A-5C), while the third ring 430 flexes to reposition the second ring 420. In various embodiments, the second ring 420 contacts the outward chamfer 360 of the cap 130 (either initially or due to flexion in the third ring 430) or the inward chamfer 240 of the third port 230, which further directs how the self-healing seal 140 bends when under uneven pressures.

FIG. 5A shows a through-hole 510 created between the first side and the second side of the self-healing seal 140 (e.g., via inserting of an instrument 150 from the first side, shown on the left, to the second side, shown on the right). In FIG. 5A, the first side and the second side are subject to substantially equal pressures, and the self-healing seal 140 is shown in a neutral position. The inverted conic bevel 440 helps align the instrument 150 so that the through-hole 510 is substantially aligned on the centerline of the self-healing seal 140, with the first dimple 450 and second dimple 460 (not illustrated in FIG. 5A) helping to align the tear, and minimize unintended or secondary tearing. Depending on the gauge of the instrument 150, and whether the instrument 150 is hollow or solid, the through-hole 510 may have a larger or smaller cross-sectional area than is shown in FIG. 5A.

In FIG. 5B, the third ring 430 flexes inward (relative to the first passage 122) such as when a negative pressure (such as vacuum suction) is exerted by the pressure source 160 into the first passage 122. When drawn inward, the walls of the inverted conic bevel 440 are drawn together, thereby sealing the through-hole 510 (not illustrated in FIG. 5B) during operation of a pressure source 160 applying suction to a fluid target via a port adapter 100 using a punctured self-healing seal 140 (e.g., to aspirate a blood clot from a vein). In various embodiments, the second side of the third ring 430 contacts the inward chamfer 240 of the third port 230, which directs how the self-healing seal 140 flexes inward, thereby encouraging the third ring 430 and second ring 420 to move so as to collapse centrally inward to seal the through-hole 510 when a threshold negative pressure is exerted on the second side of the self-healing seal 140. In various embodiments, the threshold negative pressure is smaller in magnitude than the applied negative pressure (e.g., −X psi when the pressure source applies-n*X psi to the fluid target, where n is a negative pressure safety factor such as 1.1, 1.5, 2, 3, or the like chosen by the designer).

In FIG. 5C, the third ring 430 flexes outward (relative to the first passage 122) such as when a positive pressure is exerted by the pressure source 160 into the first passage 122. When pushed outward, the walls of the tearing guide 470 are drawn together, thereby sealing the through-hole 510 (not illustrated in FIG. 5B) during operation of a pressure source 160 applying positive pressure to a fluid target via a port adapter 100 using a punctured self-healing seal 140 (e.g., to inject a substance into a vein). Additionally or alternatively to the walls of the tearing guide 470 being drawn together to seal the through-hole 510, the outer edges of the second ring 420 may be pushed centrally by the outward chamfer 360 of the cap 130, allowing at least a portion of the inverted conic bevel 440 to be pushed together to seal the through-hole 510, thereby encouraging the third ring 430 and second ring 420 to move so as to collapse centrally inward to seal the through-hole 510 when a threshold positive pressure is exerted on the second side of the self-healing seal 140. In various embodiments, the threshold positive pressure is smaller in magnitude than the applied positive pressure from the pressure source 160 (e.g., −Y psi when the pressure source applies −p*Y psi to the fluid target, where p is a positive pressure safety factor such as 1.1, 1.5, 2, 3, or the like chosen by the designer).

In various embodiments, the designer may select different materials for the self-healing seal 140, adjust the absolute and relative circumferences of the various rings 410/420/430 (e.g., in the ZY plane), and adjust the absolute and relative thicknesses of the various rings 410/420/430, to affect how readily (e.g., at what threshold pressure) the self-healing seal 140 closes a through-hole 510 when a pressure differential is applied to the self-healing seal 140. Additionally or alternatively, the designer may change where the edges of the through-hole in the cap 130 (e.g., the 360) are located relative to the diameter of the second ring 420 to position the cap 130 to contact the raised ridge sooner or later when the self-healing seal 140 flexes outward. Additionally or alternatively, the designer may change where the edges of the third port 230 (e.g., adjusting the fifth bores B5, sixth bore B6, second angle A2, and combinations thereof) are mated relative to the second side of the self-healing seal 140 to make and break contact with the third ring 430 sooner or later when the self-healing seal 140 flexes inward.

The present disclosure may also be understood with reference to the following numbered clauses.

Clause 1: A device, comprising: a body including a first port, a second port, and a third port; a cap, secured to the third port; and a self-healing seal secured to the third port by the cap, wherein the self-healing seal has a circular face and includes: a first ring located on an edge of the circular face, captured between the body and the cap; and a second ring, located centrally on the circular face, including a raised ridge on a first side of the circular face defining an inverted conic bevel, and including a tearing guide on a second side of the circular face centered on the inverted conic bevel.

Clause 2: The device of any of clauses 1 and 3-11, wherein: the first port is aligned on a shared axis with the third port; and the second port is disposed between the first port and the third port at an intersecting angle with the shared axis.

Clause 3: The device of any of clauses 1, 2, and 4-11, wherein the third port includes an inward chamfer where mated to the self-healing seal.

Clause 4: The device of any of clauses 1-3 and 5-11, wherein the cap includes a through-hole of a bore sized relative to a diameter of the second ring, wherein edges of the through-hole are positioned to contact the raised ridge when a positive pressure is applied to the second side of the circular face.

Clause 5: The device of any of clauses 1-4 and 6-11, wherein the self-healing seal includes a third ring of reduced thickness relative to the first ring and the second ring located between the first ring and the second ring.

Clause 6: The device of any of clauses 1-5 and 7-11, wherein the self-healing seal is configured, when a through-hole is formed between the first side and the second side between a nadir of the inverted conic bevel and the tearing guide, to collapse the inverted conic bevel to seal the through-hole when a negative pressure is applied from the second port to the first port.

Clause 7: The device of any of clauses 1-6 and 8-11, wherein the self-healing seal is configured, when a through-hole is formed between the first side and the second side between a nadir of the inverted conic bevel and the tearing guide, to collapse the tearing guide to seal the through-hole when a positive pressure is applied from the second port to the first port.

Clause 8: The device of any of clauses 1-7 and 9-11, wherein when a through-hole is formed between the first side and the second side of the self-healing seal, the circular face is configured to collapse inward in a direction of a lower pressure to maintain a seal between the first side and the second side for pressure differentials up to 40 pounds per square inch as applied across the first side and the second side of the circular face.

Clause 9: The device of any of clauses 1-8, 10, and 11, further comprising: a Luer activated valve disposed in a fourth port of the body.

Clause 10: The device of any of clauses 1-9 and 11, further comprising: a second self-healing seal secured to the self-healing seal by the cap, wherein the second self-healing seal has a circular face and includes: a first ring located on an edge of the circular face, captured between the body and the cap; and a second ring, located centrally on the circular face, including a raised ridge on a first side of the circular face defining an inverted conic bevel, and including a second tearing guide on a second side of the circular face centered on the inverted conic bevel; wherein the second self-healing seal is concentrically aligned with the self-healing seal on a shared axis between the tearing guide and the second tearing guide; and wherein the second self-healing seal and the self-healing seal form an airlock between the first side of the circular face of the second self-healing seal and the second side of the circular face of the self-healing seal.

Clause 11: The device of any of clauses 1-10, wherein when a through-hole is formed between the first side and the second side of the self-healing seal and the first side and the second side of the second self-healing seal, the circular face of the self-healing seal and the circular face of the second self-healing seal are configured to collapse inward in a direction of a lower pressure to maintain a seal between the first side of the circular face and the second side of the second circular face for pressure differentials up to 80 pounds per square inch as applied across the first side of the circular face of the self-healing seal and the second side of the circular face of the second self-healing seal.

Clause 12: A self-healing seal, comprising: a first ring located on an edge of a circular face; and a second ring, located centrally on the circular face, including a raised ridge on a first side of the circular face and defining an inverted conic bevel, and including a tearing guide on a second side of the circular face centered on the inverted conic bevel.

Clause 13: The self-healing seal of any of clauses 12 and 14-19 wherein the tearing guide is a cavity centered on the inverted conic bevel and having radius smaller than the second ring, the tearing guide including a first nadir aligned with a second nadir of the inverted conic bevel at a thinnest point of the self-healing seal.

Clause 14: The self-healing seal of any of clauses 12, 13, and 14-19, wherein when a through-hole is formed between the first side and the second side and negative pressure is applied to the second side, the second ring is configured to collapse the inverted conic bevel inward to seal the through-hole.

Clause 15: The self-healing seal of any of clauses 12-14 and 16-19, wherein when a through-hole is formed between the first side and the second side and positive pressure is applied to the second side, the tearing guide is configured to collapse inward to seal the through-hole.

Clause 16: The self-healing seal of any of clauses 12-15, and 17-19, wherein when a through-hole is formed between the first side and the second side, the circular face is configured to collapse inward in a direction of a lower pressure to maintain a seal between the first side and the second side for pressure differentials up to 40 pounds per square inch as applied across the first side and the second side of the circular face.

Clause 17: The self-healing seal of any of clauses 12-16 and 18-19, wherein the first ring is configured to interface with a ring of a second self-healing seal to define an airlock between the second side of the circular face and the second self-healing seal when the first ring is concentrically aligned with the ring.

Clause 18: The self-healing seal of any of clauses 12-17 and 19, wherein the first ring is configured to interface with a ring of a second self-healing seal to define an airlock between the first side of the circular face and the second self-healing seal when the first ring is concentrically aligned with the ring.

Clause 19: The self-healing seal of any of clauses 12-18, further comprising a puncture slit defined on and located centrally on the circular face, defining a region of reduced thickness in the self-healing seal to reduce tearing of the circular face outside of the region when forming a through-hole.

Clause 20: A device, comprising: a body including a first port, a second port, and a third port; a sealing means connected to the third port that is configured to maintain a seal when a pressure is applied to the first port through the second port, the sealing means including: a first ring located on an edge of a circular face; and a second ring, located centrally on the circular face, including a raised ridge on a first side of the circular face that defines an inverted conic bevel, and includes a tearing guide on a second side of the circular face centered on the inverted conic bevel.

Clause 21: The device of any of clauses 20 and 22-31, wherein a nadir of the inverted conic bevel is located at a height between a peak of the raised ridge and a foot of the raised ridge.

Clause 22: The device of any of clauses 20, 21, and 23-31, wherein a peak of the raised ridge matches a peak of the first ring.

Clause 23: The device of any of clauses 20-22 and 24-31, wherein: the first port is aligned on a shared axis with the third port; and the second port is disposed between the first port and the third port at an intersecting angle with the shared axis.

Clause 24: The device of any of clauses 20-23 and 25-31, wherein the third port includes an inward chamfer where mated to the sealing means.

Clause 25: The device of any of clauses 20-24 and 26-31, wherein the sealing means includes a cap that includes a through-hole of a bore sized relative to a diameter of the second ring, wherein edges of the through-hole are positioned to contact the raised ridge when a positive pressure is applied to the second side of the circular face.

Clause 26: The device of any of clauses 20-25 and 27-31, wherein the sealing means includes a third ring of reduced thickness relative to the first ring and the second ring located between the first ring and the second ring.

Clause 27: The device of any of clauses 20-26 and 28-31, wherein the sealing means is configured, when a through-hole is formed between the first side and the second side between a nadir of the inverted conic bevel and the tearing guide, to collapse the inverted conic bevel to seal the through-hole when a negative pressure is applied from the second port to the first port.

Clause 28: The device of any of clauses 20-27 and 29-31, wherein the sealing means is configured, when a through-hole is formed between the first side and the second side between a nadir of the inverted conic bevel and the tearing guide, to collapse the tearing guide to seal the through-hole when a positive pressure is applied from the second port to the first port.

Clause 29: The device of any of clauses 20-28, 30, and 31, further comprising: a Luer activated valve disposed in a fourth port of the body.

Clause 30: The device of any of clauses 20-29 and 31, wherein the sealing means further comprises: a third ring located on a second edge of a second circular face; and a fourth ring, located centrally on the second circular face, including a second raised ridge on a first side of the second circular face that defines an second inverted conic bevel, and includes a second tearing guide on a second side of the second circular face centered on the second inverted conic bevel; wherein the first side of the second circular face and the second side of the circular face form an airlock; and wherein the second ring and the fourth ring are coaxially aligned.

Clause 31: The device of any of clauses 20-30, wherein the sealing means is configured to maintain a seal after the circular face and the second circular face are punctured when a pressure differential of 80 pounds per square inch is applied across the first side of the circular face and the second side of the second circular face.

The descriptions and illustrations of one or more embodiments provided in this disclosure are intended to provide a thorough and complete disclosure the full scope of the subject matter to those of ordinary skill in the relevant art and are not intended to limit or restrict the scope of the subject matter as claimed in any way. The aspects, examples, and details provided in this disclosure are considered sufficient to convey possession and enable those of ordinary skill in the relevant art to practice the best mode of the claimed subject matter. Descriptions of structures, resources, operations, and acts considered well-known to those of ordinary skill in the relevant art may be brief or omitted to avoid obscuring lesser known or unique aspects of the subject matter of this disclosure. The claimed subject matter should not be construed as being limited to any embodiment, aspect, example, or detail provided in this disclosure unless expressly stated herein. Regardless of whether shown or described collectively or separately, the various features (both structural and methodological) are intended to be selectively included or omitted to produce an embodiment with a particular set of features. Further, any or all of the functions and acts shown or described may be performed in any order or concurrently.

Having been provided with the description and illustration of the present disclosure, one of ordinary skill in the relevant art may envision variations, modifications, and alternate embodiments falling within the spirit of the broader aspects of the general inventive concept provided in this disclosure that do not depart from the broader scope of the present disclosure.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, and A-B-C, where the sets may include one or multiple instances of a given member (e.g., A-A, A-A-A, A-A-B, A-A-B-B-C-C-C, etc.) and any ordering thereof.

As used in the present disclosure, the term “determining” encompasses a variety of actions that may include calculating, computing, processing, deriving, investigating, looking up (e.g., via a table, database, or other data structure), ascertaining, receiving (e.g., receiving information), accessing (e.g., accessing data in a memory), retrieving, resolving, selecting, choosing, establishing, and the like.

As used in the present disclosure, the terms “substantially”, “approximately”, “about”, and other relative terms encompass values within ±5% of a stated quantity, percentage, or range unless a different approximation is explicitly recited in relation to the state quantity, percentage, or range or if the context of the value indicates that a different approximation would be more appropriate. For example, a value identified as about X % may be understood to include values between 0.95*X % and 1.05*X % or between X−0.05X and X+0.05X percent, but may stop at zero or one hundred percent in various contexts. In another example, a feature described as being substantially parallel or perpendicular to another feature shall be understood to be within +9 degrees of parallel or perpendicular. Any value stated in relative terms shall be understood to include the stated value and any range or subrange between the indicated or implicit extremes.

As used in the present disclosure, all numbers given in the examples (whether indicated as approximate or otherwise) inherently include values within the range of precision and rounding error for that number. For example, the number 4.5 shall be understood to include values from 4.45 to 4.54, while the number 4.50 shall be understood to include values from 4.495 to 4.504. Additionally, any number or range that explicitly or by context refers to an integer amount (e.g., approximately X users, between about Y and Z states), shall be understood to round downward or upward to the next integer value (e.g., X+1 users, Y−1 and Z+1 states).

The following claims are not intended to be limited to the embodiments shown herein, but are to be accorded the full scope consistent with the language of the claims. Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various aspects described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

1. A device, comprising:
a body including a first port, a second port, and a third port;
a cap, secured to the third port; and
a self-healing seal secured to the third port by the cap, wherein the self-healing seal has a circular face and includes:
a first ring located on an edge of the circular face, captured between the body and the cap; and
a second ring, located centrally on the circular face, including a raised ridge on a first side of the circular face defining an inverted conic bevel, and including a tearing guide on a second side of the circular face centered on the inverted conic bevel.

2. The device of claim 1, wherein:
the first port is aligned on a shared axis with the third port; and
the second port is disposed between the first port and the third port at an intersecting angle with the shared axis.

3. The device of claim 1, wherein the third port includes an inward chamfer where mated to the self-healing seal.

4. The device of claim 1, wherein the cap includes a through-hole of a bore sized relative to a diameter of the second ring, wherein edges of the through-hole are positioned to contact the raised ridge when a positive pressure is applied to the second side of the circular face.

5. The device of claim 1, wherein the self-healing seal includes a third ring of reduced thickness relative to the first ring and the second ring located between the first ring and the second ring.

6. The device of claim 1, wherein the self-healing seal is configured, when a through-hole is formed between the first side and the second side between a nadir of the inverted conic bevel and the tearing guide, to collapse the inverted conic bevel to seal the through-hole when a negative pressure is applied from the second port to the first port.

7. The device of claim 1, wherein the self-healing seal is configured, when a through-hole is formed between the first side and the second side between a nadir of the inverted conic bevel and the tearing guide, to collapse the tearing guide to seal the through-hole when a positive pressure is applied from the second port to the first port.

8. The device of claim 1, wherein when a through-hole is formed between the first side and the second side of the self-healing seal, the circular face is configured to collapse inward in a direction of a lower pressure to maintain a seal between the first side and the second side for pressure differentials up to 40 pounds per square inch as applied across the first side and the second side of the circular face.

9. The device of claim 1, further comprising:
a Luer activated valve disposed in a fourth port of the body.

10. The device of claim 1, further comprising:
a second self-healing seal secured to the self-healing seal by the cap, wherein the second self-healing seal has a circular face and includes:
a first ring located on an edge of the circular face, captured between the body and the cap; and
a second ring, located centrally on the circular face, including a raised ridge on a first side of the circular face defining an inverted conic bevel, and including a second tearing guide on a second side of the circular face centered on the inverted conic bevel;
wherein the second self-healing seal is concentrically aligned with the self-healing seal on a shared axis between the tearing guide and the second tearing guide; and
wherein the second self-healing seal and the self-healing seal form an airlock between the first side of the circular face of the second self-healing seal and the second side of the circular face of the self-healing seal.

11. The device of claim 10, wherein when a through-hole is formed between the first side and the second side of the self-healing seal and the first side and the second side of the second self-healing seal, the circular face of the self-healing seal and the circular face of the second self-healing seal are configured to collapse inward in a direction of a lower pressure to maintain a seal between the first side of the circular face and the second side of the second circular face for pressure differentials up to 80 pounds per square inch as applied across the first side of the circular face of the self-healing seal and the second side of the circular face of the second self-healing seal.

12. A self-healing seal, comprising:
a first ring located on an edge of a circular face; and
a second ring, located centrally on the circular face, including a raised ridge on a first side of the circular face and defining an inverted conic bevel, and including a tearing guide on a second side of the circular face centered on the inverted conic bevel.

13. The self-healing seal of claim 12, wherein the tearing guide is a cavity centered on the inverted conic bevel and having radius smaller than the second ring, the tearing guide including a first nadir aligned with a second nadir of the inverted conic bevel at a thinnest point of the self-healing seal.

14. The self-healing seal of claim 12, wherein when a through-hole is formed between the first side and the second side and negative pressure is applied to the second side, the second ring is configured to collapse the inverted conic bevel inward to seal the through-hole.

15. The self-healing seal of claim 12, wherein when a through-hole is formed between the first side and the second side and positive pressure is applied to the second side, the tearing guide is configured to collapse inward to seal the through-hole.

16. The self-healing seal of claim 12, wherein when a through-hole is formed between the first side and the second side, the circular face is configured to collapse inward in a direction of a lower pressure to maintain a seal between the first side and the second side for pressure differentials up to 40 pounds per square inch as applied across the first side and the second side of the circular face.

17. The self-healing seal of claim 12, wherein the first ring is configured to interface with a ring of a second self-healing seal to define an airlock between the second side of the circular face and the second self-healing seal when the first ring is concentrically aligned with the ring.

18. The self-healing seal of claim 12, wherein the first ring is configured to interface with a ring of a second self-healing seal to define an airlock between the first side of the circular face and the second self-healing seal when the first ring is concentrically aligned with the ring.

19. The self-healing seal of claim 12, further comprising:
a puncture slit defined on and located centrally on the circular face, defining a region of reduced thickness in the self-healing seal to reduce tearing of the circular face outside of the region when forming a through-hole.

20. A device, comprising:
a body including a first port, a second port, and a third port;
a sealing means connected to the third port that is configured to maintain a seal when a pressure is applied to the first port through the second port, the sealing means including:
a first ring located on an edge of a circular face; and
a second ring, located centrally on the circular face, including a raised ridge on a first side of the circular face that defines an inverted conic bevel, and includes a tearing guide on a second side of the circular face centered on the inverted conic bevel.

21-31. (canceled)
